(19) United States
(12) Patent Application Publication
(10) Pub. No.: US 2018/0099009 A1
(43) Pub. Date: Apr. 12, 2018

(54) Bony Tissue Delivery Systems and Methods

(71) Applicant: Rediens, Inc., Burlington, CA (US)

(72) Inventors: Francois BINETTE, Napa, CA (US); Lawrence A. SHIMP, Burlington, WI (US)

(21) Appl. No.: 15/560,132

(22) PCT Filed: Mar. 15, 2016
(86) PCT No.: PCT/US2016/022461

§ 371 Date: Sep. 20, 2017

Related U.S. Application Data
(60) Provisional application No. 62/208,760, filed on Aug. 23, 2015.
(60) Provisional application No. 62/135,835, filed on Mar. 20, 2015.

(51) Int. Cl. A61K 35/28; A61M 1/00; A61M 3/02

(52) U.S. Cl. CPC A61K 35/28; A61M 3/0262; A61M 1/007; A61M 1/0064

(57) ABSTRACT

A delivery system and method suitable for delivering a fluid to an interior of a bone and for removing fluid including bodily material from the interior of the bone. The delivery system includes a proximal portion graspable by a user and in fluid communication with a source of negative pressure and a delivery fluid. A delivery cannula has a length sufficient to extend from the proximal portion to a distal tip insertable into the interior of the bone to deliver the delivery fluid into the bone. A removal cannula has a length sufficient to extend from the proximal portion to a distal tip insertable into the interior of the bone to withdraw fluid including bodily material from the bone.

### CROSS-REFERENCE TO RELATED APPLICATION

This application claims priority to U.S. Provisional Application Nos. 62/208,760 and 62/135,835 filed on 23 Aug. 2015 and 20 Mar. 2015, respectively. The entire contents of each of the above-mentioned applications are incorporated herein by reference.

### FIELD OF THE INVENTION

This invention relates to delivery devices and methods and more particularly to those suitable for delivery of compositions within bone.

### BACKGROUND OF THE INVENTION

Chronic back pain (CBP) can be very debilitating and often does not respond well, long term, to conventional treatments such as over the counter medicine, physical therapy etc. Over time, if the pain is too disruptive and does not subside, risky invasive surgical intervention within the spine of a patient may be necessary. Furthermore, treatment of back pain is very costly to the economy in terms of actual treatment and disability costs, or loss of productivity.

The spine includes bony vertebral bodies separated by soft intervertebral discs (IVD) that permit relative movement of the vertebral bodies and give the spine its flexibility. The IVDs are made of two distinct structures, the nucleus pulposus (NP) a soft gelatinous core surrounded by a dense fibrous shell called the annulus fibrosus (AF). IVD are almost completely avascular and devoid of innervation. IVDs can fail by progressive extrusion where the NP or AF spreads outward beyond the area of the vertebral bodies and impinges on the spinal nerves. The discs can also rupture where the walls fail and the NP leaks out from an IVD.

Vertebral bodies have a cortical (dense) bone shell several mm thick surrounding a core of honeycombed bone (cancellous bone) filled with marrow. The surfaces of the vertebral bodies resting on the discs are called end plates. Vertebral bodies can fracture both in their vertical walls and in their end plates. Inside, the cancellous bone can resorb, thereby increasing the chances of fracture, and the marrow can become fatty and inflamed.

The predominant cause of CBP is the progressive degeneration of the IVD. The causes of IVD degeneration are complex and include: injuries, genetics, nutrition, mechanical stresses, smoking, and numerous other factors. Over time, the degenerating IVD will lose its mechanical integrity causing it to collapse. The resulting collapsing disc causes the structural parts of the spine to impinge on the spinal nerves and causes acute pain. Collapse of the vertebral bodies can also lead to nerve impingement issues and acute back pain.

There are also many cases of spinal pain where direct nerve impingement is not an issue, and the pain is coming from the spine itself. This is called axial or discogenic pain.

In general, spinal discs do not “feel” pain because there are little to no nerves within the discs. But the vertebral bodies, like all bone, do contain nerves and can become painful. Pain within vertebral bodies is often associated with disc degeneration and may be mitigated by procedures that repair or remove the damaged disc. There are no approved interventions for treating damaged discs that would slow down or reverse degeneration. When discogenic pain becomes intractable, unpredictable and invasive surgery can be prescribed to remove the disc and fuse the spine at that location to add stability. Although surgery can provide additional stability, because the actual source of pain is not addressed, spine surgery for axial back pain is often not successful and pain often returns.

Fractured or collapsed vertebral bodies as a result of weakened bones, typically resulting from conditions such as osteoporosis, are also painful on their own. Invasive therapies such as kyphoplasty or vertebroplasty that repair the immediate damage by stabilizing the fractured bone can relieve pain.

Some of most severe and intractable spinal pain comes from inflammation within the vertebral bodies. The inflammation in the bone or marrow can be visualized by Magnetic Resonance Imaging (MRI). Marrow edema or inflammation in the vertebral body is characterized by MRI by Modic changes (named after Dr. Modic who first described these changes). With the initial stage of Modic changes, the vertebral body still looks normal outside, but the areas inside are damaged and remodeling. Type 1 Modic changes are where inflammation arises in the marrow space. This can progress to Type 2 changes where the blood-making red marrow is replaced by fatty tissue, which is still inflamed. Type 3 changes are characterized by remodeling into abnormal sclerotic bone, which can lead to structural anomalies and even fractures.

Inflammation is very damaging to tissues. Its biological purpose is to treat injuries and infection by chemically removing damaged tissues or invading pathogens. Long-term inflammation causes degradation of the damaged tissue, while the over-secretion of inflammatory chemicals can damage normal tissue, and irritate over-sensitize nerve endings in the bone. This leads to an ever widening zone of tissue damage and pain. The exact cause of the Modic changes is not known. End plate changes resulting in compromised integrity between the bone and the IVD have been hypothesized as a possible cause. The exposure of inflammatory disc material to the marrow in the vertebral body could activate blood cells and initiate an inflammatory process.

Another possible cause of Modic changes is bacterial infection. Propionibacterium acnes family is believed to be responsible for this damage. These bacteria are anaerobic, meaning that they require no air (oxygen) to grow and propagate. There is little or no blood in the discs themselves so this is a perfect oxygen-free environment for the bacteria to grow and multiply. They are found only in damaged discs, not healthy discs. The reason seems to be that when a disc is damaged, the body's attempts to repair it bring a temporary blood supply to the disc. This blood supply is used to supply cells that are involved in an attempt to repair disc structures. But the blood can also bring in some circulating Propionibacterium acnes bacteria that lodge in the disc and grow in the relatively low oxygen environment away from the blood vessels. Once established in the disc, these bacteria can enter the vertebral bodies through the compromised endplates and activate the blood cells from the marrow. Disc tissue can penetrate into or even through the endplates by the formation of Schmorl's nodes (small pockets of disc protrusion) and deliver bacteria that way. Antibiotics can treat the problem, but the effects are slow. About a three-month course of antibiotics typically is followed by about nine months of healing before the pain finally dissipates.

In about 40% of Modic changes there is no obvious bacterial involvement and antibiotics do not provide relief. Surgical intervention does not lead to good outcome because there are no anatomical instability or nerve impingement involved in axial or discogenic pain which can be address with a surgical procedure alone. When surgery is performed, pain often recurs after a few years.

In addition to the spine, other joints can undergo similar degenerative changes. A typical joint has a cartilage pad between the ends of two long bones. Long bones consist of an elongated cortical bone tube with a cortical wall thickness of up to 20 mm, but the bone ends that impinge on the cartilage pads are very similar to vertebral bodies in that they have a thin cortical wall enclosing a cancellous, marrow containing, honeycomb structure.

Arthritis is similar to disc degeneration because it starts with the deterioration of the cartilage pads between the bones. As with the spine, pain in the joint is felt in the bone and not in the cartilage because this tissue is completely devoid of nerves. The actual mechanism of joint deterioration in arthritis is different than in disc disease, but many of the changes in the bone are similar. In fact, marrow changes (edema) have been identified in the bone associated with painful arthritic joints. The most likely places for these changes to occur are in the hip, knee, elbow, shoulder, and ankle (talus) but changes in any bone can, in principle, be targeted.

Another joint problem is necrosis where the blood supply to the cancellous bone adjacent to the joints is cut off. This causes death of the marrow and bone, weakening the bone, and leading to fractures. Certain hip fractures, for example, are associated with necrosis of the femoral head. The usual cause of such necrosis is trauma, but in a few percent of cases, an inflammatory mechanism seems to be involved.

In many cases of bone marrow inflammation associated with disc degeneration and back pain, or with cartilage degeneration and joint pain, the structure and strength of the bone tissue is compromised. The main reason for this is an increase in extracellular matrix degrading enzymes such as various matrix metallo-proteases (MMP). Normally the role of these enzymes is to rid tissues from damaged matrix proteins as a result of an injury, but in case of chronic inflammation, these enzymes are simply too abundant and start degrading normal non-damaged tissue matrix. Weakened bone matrix resulting from a chronic inflammation could loose its integrity and collapse, compromising the bone-soft tissue boundary. Soft tissue migrating into the bone compartment can result in additional acute pain and increased inflammation compounding chronic pain.

Existing options for treating inflammation all have deficiencies. Over-the-counter inflammation treatments are not effective at all for Modic conditions. In cases of acute back pain from disc herniation extruding and impinging on nerves, injection of corticosteroids in the perispinal epidural space can help. These anti-inflammatory agents provide nearly instant relief, but have short duration and they have potentially serious side effects and long-term use can be risky. Corticosteroid injections are not indicated for axial or discogenic chronic back pain. Other anti-inflammatory agents have not been injected directly in the inflamed marrow of the bone. A surgical method of treating inflammation by removing or ablating the inflammatory agents and damaged tissue could be envisioned. This is not easy, however, and it is very difficult to provide complete removal of inflamed marrow within a closed bone space. Removing inflamed tissue is not expected to provide long-term benefit since it does not address the underlying cause of inflammation. So, while some relief may be obtained, the inflammation will return later. More drastic surgical techniques such as spinal fusion can provide relief, but often it is temporary and results are unpredictable since there are no obvious anatomical deficiencies or instability to begin with. In other anatomical areas, replacement of an arthritic joint with an artificial joint provides permanent relief because all of the damaged bone is removed. Because of the extensive trauma and associated risks, and high costs from invasive spine, or other joint replacement surgeries, a method for avoiding or delaying orthopedic surgery would be of great benefit.

It is therefore desirable to have improved delivery devices and methods to treat chronic back pain and other ailments associated with bony tissues.

### SUMMARY OF THE INVENTION

An object of the present invention is to provide improved delivery devices and methods suitable for injecting compositions into bone.

Another object of the present invention is to provide less invasive techniques for treating pain arising from bony tissues.

Yet another object of the present invention is to decrease inflammation in bone.

This invention results from the realization that injection of inflammation-modulating cells into bony tissue can decrease or preferably eliminate the inflammation in the bone adjacent to a disc, or other joint cartilages such as in knee joints and hip joints, and thereby reduce or eliminate the source of chronic pain. Methods according to the present invention seek to eliminate inflammation without relying on removal of the inflamed tissue (though such removal can be used in conjunction with the treatment) and without significant side effects.

This invention features novel delivery devices and methods suitable to treat inflammation within the marrow space of bones. Enabling direct treatment of inflammation from injecting compositions including inflammation-modulating cells, it can provide a sustained relief from pain and, by eliminating the inflammation, it can allow the bone to heal and regain its former strength. Since the compositions are introduced through a needle directly into the bone, it does not require invasive risky surgery and therefore greatly lowers the risk of adverse events. Moreover, unlike corticosteroids, steroids and other drug injections which provide only short term solutions with significant risks of side effects, the injection of inflammation modulating cells is expected to have little risk and a prolonged benefit.

This invention may be expressed as a delivery system and method suitable for delivering a fluid to an interior of a bone and for removing fluid including bodily material from the interior of the bone. The delivery system includes a proximal portion graspable by a user and in fluid communication with a source of negative pressure and a delivery fluid. A delivery cannula has a length sufficient to extend from the proximal portion to a distal tip insertable into the interior of the bone to deliver the delivery fluid into the bone. A removal cannula has a length sufficient to extend from the proximal portion to a distal tip insertable into the interior of the bone to withdraw fluid including bodily material from the bone.

The treatment can be used in conjunction with conventional physical augmentation techniques such as grafting with a natural bone material, or with a synthetic scaffold material or a reinforcing material.

### BRIEF DESCRIPTION OF THE DRAWINGS

In what follows, preferred embodiments of the invention are explained in more detail with reference to the drawings, in which:

FIG. 1 is a schematic side, partial cross-sectional view of a distal portion of a delivery device according to the present invention inserted into a vertebral body of a patient;

FIG. 1A is a view similar to FIG. 1 showing a proximal portion of one device construction according to the present invention

FIG. 2 is a schematic perspective view of a distal portion of a device according to the present invention having parallel fixed or sliding cannulas;

FIG. 3 is a view similar to FIG. 2 showing concentric cannulas;

FIG. 4 is a schematic illustration of a device according to the present invention having a single plunger to simultaneously deliver compounds or irrigation fluids and withdraw bodily material such as inflamed or damaged tissue;

FIG. 5 is a schematic perspective view of a dual chamber syringe having a suction chamber larger than a delivery chamber plus fixed inner and outer cannulas;

FIG. 6 is a view similar to FIG. 5 with an outer suction cannula that moves axially relative to a fixed inner cannula;

FIG. 7 is a schematic perspective view of another delivery device according to the present invention having a control valve which enables selective control of delivery and suction; and

FIGS. 8A-8C are enlarged schematic cross-sectional views of one construction of the control valve of FIG. 7.

### DETAILED DESCRIPTION OF THE PRESENTLY PREFERRED EMBODIMENTS

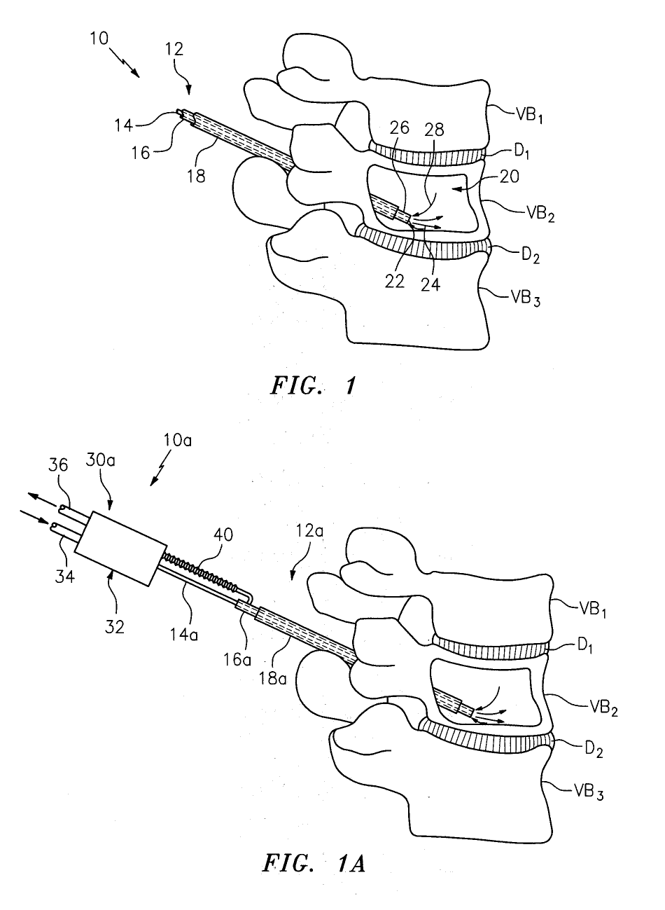

Delivery devices according to the present invention are described in more detail below in relation to FIGS. 1-8C. The anti-inflammatory treatment of this invention consists of injecting or otherwise placing certain cell types in bone suspected of having an inflamed area. If cells that are known for being anti-inflammatory agents are placed in an inflamed site within bone according to the present invention, the inflammation decreases almost immediately. This is regardless of the cause of the inflammation. The exact mechanism by which inflammation is suppressed is unknown, but cytokines and other secretory bio-molecules known to affect inflammation and inflammation signalling are suspected to play an important role. Examples of cells with anti-inflammatory properties are mesenchymal stem cells (MSCs), derived from bone marrow, stromal vascular stem cells, and any engineered or isolated cells yet to be developed that have appropriate anti-inflammatory properties.

One key advantage of using living cells as the anti-inflammatory agent is that the cells are used as miniature bioactive manufacturing systems, which upon implantation will provide a sustained and long-term production of those anti-inflammation bio-molecules. Certain types of stem cells such as MSCs are further known to be immune-privileged, meaning that they can evade immune rejection for a while, conferring them with an even longer benefit. This also means that allogenic MSCs (cells from another person) can be effective as well as autogenic or autologous cells (cells from the patient). Longer lasting cells will provide longer-term bioactive secretion modulating or decreasing inflammation-causing pain. Living cells such as MSCs can also secrete combinations of inflammation modulating biomolecules, which can act in additive or synergistic fashion to provide better activity than purified anti-inflammatory drugs.

Even if the inflammation is secondary to another cause such as infection or a tissue injury, it is still advantageous to treat the inflammation as well as the primary cause. As was mentioned earlier, one possible source of inflammation in diseased spines is a bacterial infection. Although antibiotics can treat infections, it can take up to a year to finally get relief. The invention can give relief at the same time the antibiotic treatment is started, or anytime during and after the course of the antibiotic treatment, in cases where an infection is the suspected cause of the inflammation.

Cells utilized according to the present invention can be sourced from the patient (autogenic or autologous) or from another person (allogenic). For safety, the patient's own cells are preferred since there is little to no risk of transmitting diseases that the patient has not been already exposed to. Collecting the cells intraoperatively avoids the costs and problems associated with keeping the cells alive outside of the body for an extended period. The most abundant source of such autogenic cells are the MSCs which are found in many tissues including bone marrow, circulating in blood, and also in fat deposits (adipose tissue). Harvesting these cells can be carried out by a variety of means (usually involving aspiration), but they are always initially mixed with other cells. It is desirable to separate or enrich the MSCs from other cells as much as possible in order to get a more predictable effect. There are several means of doing so, involving, for example, antibodies, enzymes, mechanical means, or a combination of methods. Purely mechanical means, especially by centrifugation, are preferred since there are no foreign substances introduced.

Alternatively, for added convenience, and enhanced bioactivity, cells that have been isolated from a donor(s), amplified with various tissue or cell culture techniques, banked, and qualified for their ability to tame inflammation could be used. Such cells can be prepared in very large batches and frozen into therapeutic doses until needed for use. At the time, the health provider can thaw the cells and prepare for delivery. Large cell banks can be carefully studied and selected for their potency, absence of adventitious agents and provide for safe and effective cell source at the point of care without the need for the additional procedures of harvesting donor bone marrow or adipose tissue and a time consuming processing of isolating and enriching MSCs before injections.

During the process of propagation and selection of allogenic cells, further manipulation could be considered which would provide additional benefits or enhanced anti-inflammatory activity. For example, MSC or other cells could be engineered by introducing a gene known to modulate inflammation. The product of such a gene could be a cytokine or hormone that would act locally on the inflamed tissue, or remotely by recruiting and signalling the host immune cells to migrate to the inflammation area and decrease the inflammation. Many ways of introducing genes in cells are known to people who are skilled in the art and include but are not limited to: lipid mediated transfer, viral transduction, chemical transfection and electroporation.

The cells whether autologous or allogenic can be delivered to the site of inflammation as suspension by means of a cannula such as a hollow hypodermic needle or a catheter, as discussed in more detail below. Open surgery is also possible, but it is much less desirable. The cells can also be delivered suspended in a formulation, which would enhance their viability through the injection procedure or post-injection at the site of implantation. Such a formulation could include many different soluble chemical preparations maintaining proper physiological condition prior, during or after the injection. Furthermore, the formulation could also include alone or in combination with various soluble factors, soluble or insoluble biomaterials which would act at minimizing sheer stress on the cells during the injection, or help stabilize the cells prior to, or after the injection. Formulations according to the present invention can include cell carriers such as one or more of collagen, gelatin, hyaluronic acid, saline or Ringer's solution, Agar, or similar solutions or physical augmentation material. Such a formulation could also be helpful in maintaining cell viability while the cells are frozen for storage and transportation. Formulation with various soluble and insoluble factors and biomaterials could also facilitate injection and help in the retention of the cells at the site optimal for treatment without loss due to dilution because of the presence of various body fluids such as blood, serum etc, or surgical fluids such as saline washes. Certain formulations could include natural bone graft fragments, a synthetic scaffold set in situ to further increase retention at the injection site. Additional formulations could include biomaterials to improve injection and retention and include only the secreted factors from the cells either purified or not, enriched or concentrated or not without the cells themselves, combined with the formulation for injection. Additional therapeutic agents can be added such as antibiotics, anti-inflammatory compounds, nano silver and/or ionic silver, and/or marker material to enhance visualization via fluoroscopy or other imaging or visualization technique.

Certain formulations in conjunction with conventional physical augmentation techniques could be injected in a specific sequence to allow mechanical stabilization of certain bone structures such as the end plate or the vertebral pedicle, or to plug a needle or trocar access point to the bone, once the delivery of the therapy is complete. The inflammation modulating cells could be injected immediately after, or just before the delivery of the conventional physical augmentation technique.

Physical augmentation material can include scaffold material including one or more of DBM (demineralized bone material), collagen, bone particles, synthetic particles such as PEEK (polyether ether ketone), calcium phosphate cement. Traditional bone cement generally may have a negative impact on cells, but could be injected after cleaning out inflammation residues and before cells are injected according to the present invention. It may be desirable to increase porosity of the cement by combining it with gelatin fibers, fast resorbing PLA (polylactic acid) fibers, or water-soluble glass such as described by James Walls in U.S. Pat. No. 8,673,018.

In cases where the treatment site is also physically compromised (such as a fractured vertebral body), conventional physical augmentation techniques can be used in conjunction with, or before or after the cells are delivered. Even if there is no mechanical/physical compromise to the treatment site, an augmentation material can be used before, in conjunction with, or after the cells are delivered. It is preferable, of course, that the augmentation technique and material is compatible with cells so as not to render the cells non-viable. For example, if an acrylic bone cement is used, then the cells should not be inserted until the cement mass has cooled below about 40 degrees C. With more biocompatible materials, the cells can be formulated with the material (such as a calcium phosphate cement) as described above and injected together, or the biomaterial can be implanted before or after the cells are injected. The cells could also be modified to incorporate a tracking dye, imaging enhancer/tracer, or gene or other reporter which could facilitate traceability, localization or persistence once injected.

Cells can be delivered to the site with little or no preparation of the site, or with more extensive preparation. Certainly, in the case of infection it is desirable to eliminate the infection before the cells are delivered. It is also desirable to eliminate as much of the inflamed tissue as possible by physically removing it by, for example, suction or washing. The method chosen cannot be 100% precise and efficient because it is not possible to image the inflamed area during the procedure. MRI is the standard detection method, but it is unsuitable for use during an operative procedure. So any method needs to be robust. However, MRI can provide a guide to the location and volume of inflamed tissue to be removed.

It is desirable if the aspiration apparatus allows a sample of the inflamed tissue to be collected for examination and characterization. Such characterization may allow a better understanding of the cause of the disease and provide information that can be used augment the basic anti-inflammatory treatment.

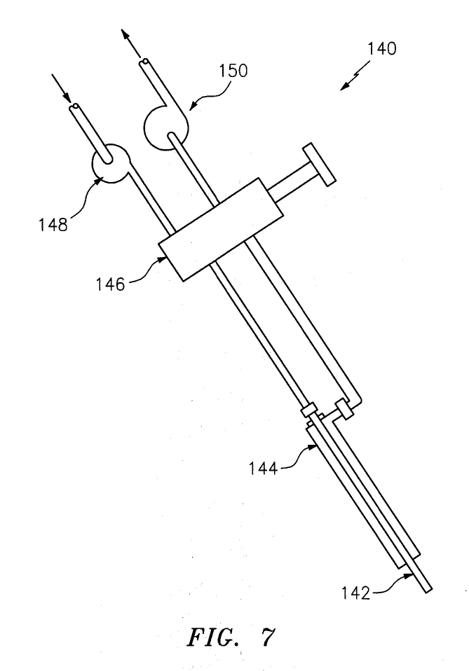

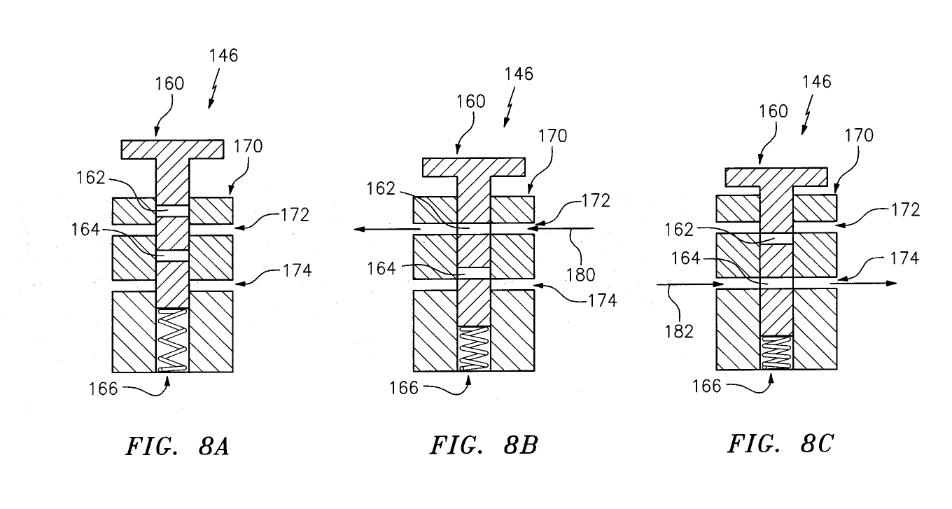

One cleaning method is to use an aspiration needle and a delivery needle to alternately suck out the inflamed marrow utilizing the aspiration needle and rinse it with saline utilizing the delivery needle. Pre-shaped needles, made of nitinol (memory alloy) in some constructions, may facilitate the procedure. The equipment may also be combined into a single apparatus with dual needles and chambers such that a flow can be set up with saline injection occurring simultaneously with marrow aspiration. A continuous flow system can also be developed where the syringe chambers are replaced with pumps and reservoirs, as described in more detail below regarding FIGS. 7-8C. A system where the second needle deploys such that it is facing the first needle may facilitate, especially, a continuous flow technique.

Preferably, devices according to the present invention are suitable to both remove fluids including particulate matter, especially inflammatory materials, from bone such as a vertebral body as well as to deliver fluids including formulations with living cells, anti-inflammatories, augmentation materials, and/or antimicrobial compositions including antibiotics. It is preferable to be able to access most of the interior of the vertebral body with flushing fluids, suction, and delivery of therapeutic materials. Several constructions for accomplishing this will be detailed below. If a needle of a delivery device according to the present invention is inserted directly in the bone it is unlikely that the needle can be moved around much inside the vertebral body so flushing would probably depend on a strong flow of saline that would extend well beyond the end of the delivery needle to loosen and liquefy the “bad” materials. For maximum effectiveness, the suction should be shut off during the saline delivery phase.

If a guide tube, also referred to as an outer cannula, is inserted in the bone before delivery/suction devices are inserted, then the fluid deliver/suction devices could be moved around somewhat inside of the vertebral body. This could help the efficiency and possibly simultaneous suction and delivery could be used. This is especially true if the suction and delivery needles could slide relative to each other.

Initial incision and placement of a distal portion of a device according to the present invention typically would follow conventional kyphoplasty and/or vertebroplasty techniques. In certain techniques according to the present invention, individual needles or catheters may be inserted for each procedure sequentially or in parallel, thus a needle for withdrawing inflammatory components, a needle for delivering saline, a needle for the cells, etc.

A distal portion 12, FIG. 1, of a dual-cannula delivery device 10 according to the present invention is shown inserted via a transpedicular (through the pedicle) approach into the interior 20 of a vertebral body VB₂ of a patient. Vertebral body VB₂ is separated from vertebral bodies VB₁ and VB₃ by discs D₁ and D₂, respectively. An inner delivery cannula 14 and an outer withdrawal cannula 16, also referred to as removal cannula 16, are passed through a guide sleeve 18, such as a needle or catheter having an inner diameter which is greater than the outer diameter of withdrawal cannula 16. Inner delivery cannula 14 has a distal tip 22 through which fluids are delivered as indicated by arrows 24. Removal cannula 16 has a distal tip 26 which draws fluids into it as indicated by arrows 28. Guide sleeve 18 remains in position to guide the delivery cannula 14 and withdrawal cannula 16 into the interior 20 of vertebral body VB2, and then is removed at the end of the procedure. The distal portion 12 can then be moved distally and proximally relative to guide sleeve 18 to effectively clean the inside of vertebral body In another technique, the delivery cannula 14 and withdrawal cannula 16 are inserted together directly into the bone without a guide sleeve.

The proximal portion of the delivery devices according to the present invention can have various constructions as described in more detail below. Delivery device 10a, FIG. 1A, has a distal portion 12a that is similar to distal portion 12, FIG. 1, and a proximal portion 30a with a handpiece 32, inlet portion 34 communication with a delivery cannula 14a, and a removal portion 36 communicating with a removal cannula 16a such as described below in relation to FIG. 7 for a pump/pneumatic system to deliver fluids to bone and withdraw unwanted fluids and materials. A flexible section 40 enables removal cannula 16a to be moved axially relative to fixed delivery cannula 14a. Both cannulas 14a and 16a are axially movable independently relative to guide sleeve 18a.

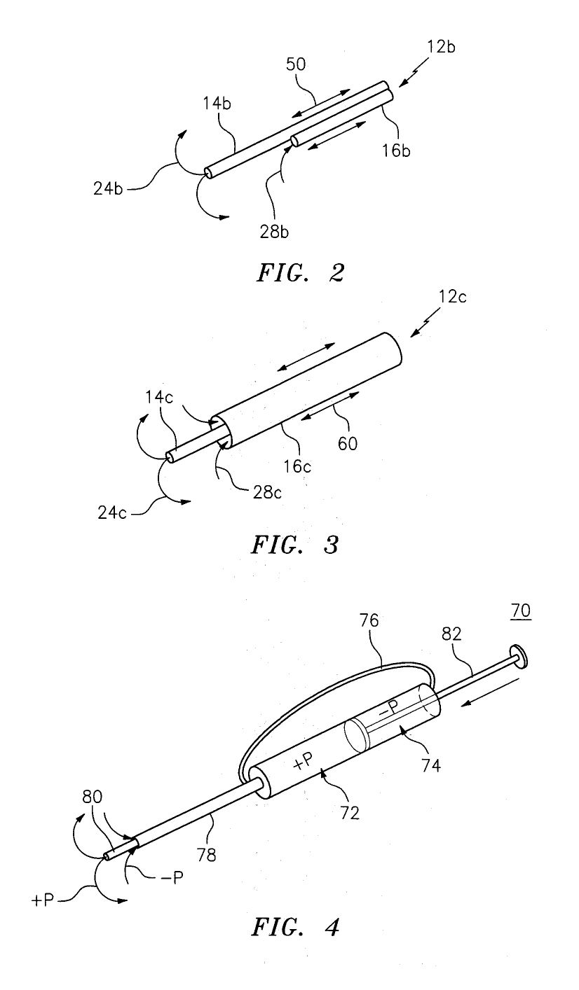

A dual-cannula device according to the present invention has a parallel configuration in some constructions and a concentric configuration in other constructions, as illustrated schematically in FIGS. 2 and 3. The geometrical relationship of the cannulas is fixed relative to each other in certain constructions or the cannulas slide independently. Distal portion 12b, FIG. 2, has parallel sliding cannulas 14b and 16b that deliver fluid, arrows 24b, or withdraw fluid, arrows 28b. Arrows 50 indicate the relative axial movement of cannulas 14b and 16b. Distal portion 12c, FIG. 3, has concentric cannulas 14c and 16c which move concentrically relative to each other as indicated by arrows 60 before, during or after delivery of fluid, arrows 24c, and removal of fluid, arrows 28c, respectively.

In certain constructions, the proximal portion includes dual chamber syringes to feed the cannulas. They would be arranged such that when a common plunger is pushed, one chamber would suck out liquid by generating negative pressure and the other chamber would feed liquid into a delivery cannula. A dual chamber syringe 70, FIG. 4, defines chambers 72 and 74 to be the same diameter and arranged in-line with each other. Arrow 76 schematically illustrates fluid including bodily material such as inflamed or damaged tissue being drawn into removal cannula 78 and then into chamber 74 when a single plunger 82 is depressed, while simultaneously delivering fluid under positive pressure from chamber 72 through delivery cannula 80 and into a patient.

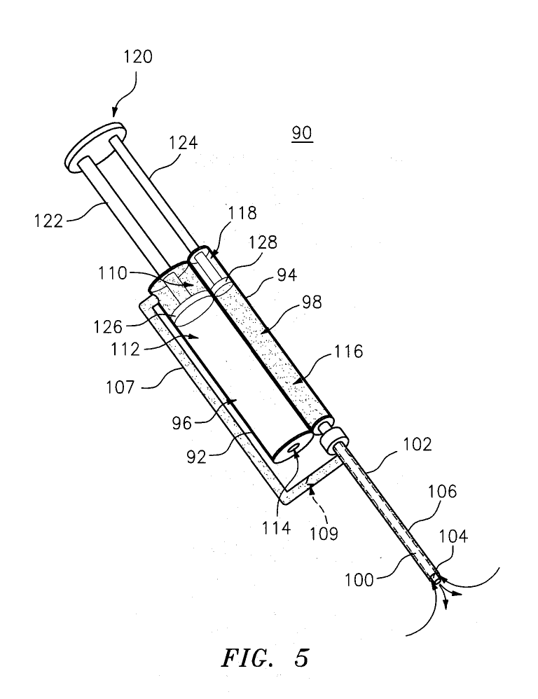

FIG. 5 is a schematic perspective view of a dual chamber syringe device 90 having parallel syringe bores 92, 94 defining a suction chamber 96 larger than a delivery chamber 98 plus fixed inner and outer cannulas 100, 102 having distal tips 104 and 106 for discharging and withdrawing fluids, respectively. Suction chamber 96 includes (1) a collection volume 110 on the proximal side of a piston 126 that communicates with withdrawal cannula 102 via a conduit 107 and (2) a gas volume 112 on the distal side of piston 126 that is filled with a gas such as air that is vented out a port 114. Delivery chamber 98 includes (1) a delivery volume 116 on the distal side of a piston 128 and (2) an empty volume 118 that communicates with the atmosphere.

As plunger 120 is depressed, plunger rods 122 and 124 transfer force to pistons 126 and 128. Fluid in delivery volume 116 is forced into delivery cannula 100 while bodily fluids and material are simultaneously aspirated into collection volume 110. In one construction, conduit 107 includes a check valve 109, shown in phantom, to inhibit backflow of fluid toward distal tip 106 of cannula 102.

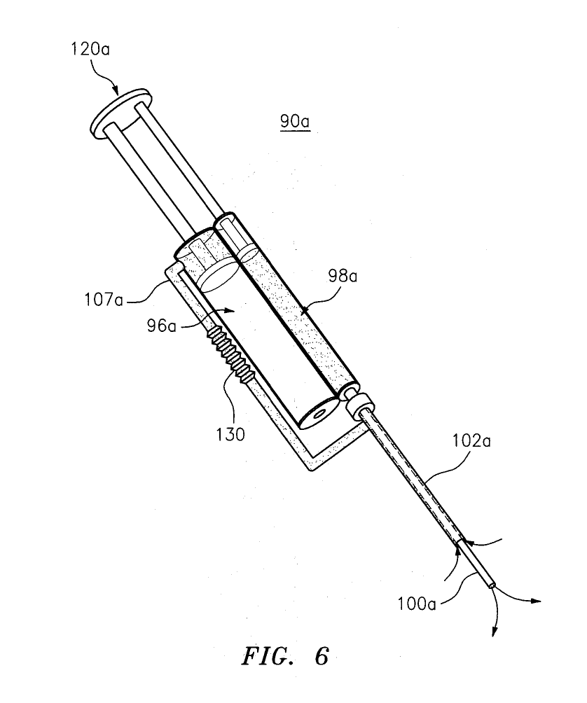

Device 90a, FIG. 6, is similar to device 90, FIG. 5, plus has the capability to axially move the removal cannula 102a relative to the delivery cannula 100a. Conduit 107a includes a flexible section 130 to enable axial movement of removal cannula 102a. A finger grip or projection can be added to the distal portion of conduit 107a or to removal cannula 102a independent of movement of plunger 120a. In some constructions, chambers 96a and 98a are the same size in diameter and volume for both the in-line and parallel chamber designs and, in other constructions, the chambers differ in size from each other. The cannulas of devices 90 and 90a can be utilized with or without guide sleeves or catheters as described elsewhere in this application.

Instead of self-contained devices shown in FIGS. 4-6, it is also possible to use separate pumps, one for suction and one for fluid delivery. The pumps could run together, but it is best to be able to control suction and delivery separately. A preferred operating sequence is to first suck out some material, then deliver a saline flush, then shut off the saline and suck it out along with loosened inflammatory material. This cycle could be repeated several times before finally injecting therapeutic material(s). Instead of pumps, a vacuum collection reservoir could be used for suction and a pressure reservoir could be used for fluid delivery. It is also possible to provide a control system that automatically cycles between fluid deliver and suction, possibly with the time intervals being settable by the surgeon.

Delivery system 140, FIG. 7, shows a pump arrangement with a manual fluid control valve 146 between a delivery pumps 148, a suction pump 150 and associated fluid reservoirs (not shown) and the delivery cannula 142 and withdrawal cannula 144. An electric switch that turns the pumps on and off as needed could also be used as well as a pneumatic control valve that controls suction and pressurized air to suction and fluid delivery reservoirs.

Manual fluid control valve 146, FIG. 7, is shown in operation in FIGS. 8A-8C. A plunger 160 defines first and second passages 162 and 164 and is biased by a spring 166 within block 170 to an “off” position as shown in FIG. 8A. Block 170 defines first and second channels 172 and 174 which are in fluid communication with removal cannula 144 and delivery cannula 142, respectively. In this construction, initial force applied to plunger 160 causes first passage 162 to align with first channel 172 to pass bodily materials as indicated by arrow 180, FIG. 8B, through suction pump 150, FIG. 7. Further depression of plunger 160 causes second passage 164, FIG. 8C, to align with channel 174, also referred to as a saline passage, to enable delivery of fluid as indicated by arrow 182.

Therapy could be delivered through the saline passage with suction turned off in the case of a free-flowing therapeutic material such as cells in a carrier or possibly a liquid self-setting calcium phosphate cement. In the case of a particulate augmentation material it may be preferred to remove the twin needle device and replace it with a larger diameter catheter.

Cells and physical augmentation product can be delivered sequentially. There may be an advantage to deliver the physical scaffold first, then the cells onto the scaffold. It is also possible to mix cells and scaffold/augmentation material together before delivery if the augmentation material is cell friendly.

Individual syringes can be used for specialized delivery (or suction) with the twin needle devices used for routine operations such as flushing. Delivery needles/catheters can have more than two passages, maybe three or four passages would be useful for some indications. But in general, it is best to use a guide catheter and separate delivery devices if the delivery needs cannot be met with a two lumen delivery device.

Any other equipment, either existing or to be developed in the future, for delivering or aspirating fluids intraoperatively, may be adapted to the technique. Also, there are many options for the operative approach. Any technique that the treating physician is comfortable with is probably satisfactory. When it comes to the spine, the most common technique is expected to be the transpedicular (through the pedicle) approach used with kyphoplasty and vertebral augmentation. Examples of other techniques are a direct lateral approach and an approach through the disc into the endplate. These examples are not meant to be limiting.

If there is obvious involvement of bone inflammation on both sides of the joint or damaged disc, then the anti-inflammatory treatment should be applied to both bone areas. Even if only one bone (or vertebral body) shows obvious inflammation, the opposite bone can be treated with the anti-inflammatory cells as a prophylactic measure.

In a preferred embodiment of this invention, a surgeon or interventional radiologist would insert a needle or small trocar in the pedicule vertebral body, and through fluoro-guidance, would push the needle/trocar until it reaches the vertebral body, guide the needle to the zone of inflammation from prior diagnostic imaging, and inject MSCs at the site of inflammation. The MSCs injection could be performed after a quantity of inflamed marrow was removed by suction from the same needle or through a different one place in the vicinity from another port or access. The surgeon or radiologist would then remove the needle(s) and standard post intervention procedures for tending the soft tissue puncture site would be follow. In another embodiment of the invention, the inflamed tissue removed from the site could be characterized for diagnostic or other clinical use.

An open technique can also be used, if desired. This might be advantageous in cases where severe infection is present and open debridement is needed to remove the infected tissue.

Although specific features of the present invention are shown in some drawings and not in others, this is for convenience only, as each feature may be combined with any or all of the other features in accordance with the invention. While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature.

It is the intention, therefore, to be limited only as indicated by the scope of the claims appended hereto. Other embodiments will occur to those skilled in the art and are within the following claims.

What is claimed is:

1. A delivery system suitable for delivering a fluid to an interior of a bone and for removing fluid including bodily material from the interior of the bone, comprising:
a proximal portion graspable by a user and in fluid communication with a source of negative pressure and a delivery fluid;
a delivery cannula having a length sufficient to extend from the proximal portion to a distal tip insertable into the interior of the bone to deliver the delivery fluid into the bone; and
a removal cannula having a length sufficient to extend from the proximal portion to a distal tip insertable into the interior of the bone to withdraw fluid including bodily material from the bone.

2. The system of claim 1 wherein the delivery fluid includes cells capable of reducing inflammation.

3. The system of claim 1 wherein the proximal portion defines a first chamber containing the delivery fluid and a second chamber for generating the negative pressure and collecting the fluid including bodily material.

4. The system of claim 3 wherein the delivery fluid is delivered into the bone at the same time that fluid including bodily material is removed from the bone.

5. The system of claim 2 wherein the delivery fluid further includes a physical augmentation agent.

6. The system of claim 1 wherein the delivery cannula and the removal cannula are insertable into a vertebral body utilizing a transpedicular approach.

7. The system of claim 1 further including a guide sleeve having an interior diameter sufficient to accommodate the delivery cannula and the removal cannula.

8. The system of claim 1 wherein the removal cannula is movable axially independent of the delivery cannula.

9. The system of claim 1 wherein the removal cannula and the delivery cannula are disposed concentrically relative to each other.

10. A method for treating at least one of inflammation and pain in a bone, comprising:
selecting a bone to be treated;
selecting at least one delivery fluid including cells capable of reducing inflammation; and
delivering the delivery fluid to an interior of the bone.

11. The method of claim 10 wherein the delivery fluid includes cells selected and qualified for their ability to repress inflammation.

12. The method of claim 10 wherein the delivery fluid further includes a physical augmentation agent.

13. The method of claim 10 further including withdrawing inflamed bodily material from the bone.

14. The method of claim 11 wherein the delivery fluid includes mesenchymal cells.

15. The method of claim 11 further including at least one additional agent such as an antibiotic, an anti-inflammatory drug, or an additional cell type.
